(19) United States
(12) Patent Application Publication
(10) Pub. No.: US 2025/0387036 A1
(43) Pub. Date: Dec. 25, 2025

(54) MEASURING BLOOD ACCELERATION AND DEACCELERATION

(71) Applicant: The Regents of the University of California, Oakland, CA (US)

(72) Inventor: Jia GUO, Yorba Linda, CA (US)

(21) Appl. No.: 19/246,437

(22) Filed: Jun. 23, 2025

Related U.S. Application Data
(60) Provisional application No. 63/663,579, filed on Jun. 24, 2024.

(51) Int. Cl. A61B 5/026

(52) U.S. Cl. CPC A61B 5/0263; A61B 2576/00

(57) ABSTRACT

A method of operating a magnetic resonance imaging (MRI) system, includes operating the MRI system to make N first measurements of N first blood transit times (BTTs) in a target, wherein the N first measurements use a first value of an imaging cutoff velocity and N second values as labeling cutoff velocities respectively for the N first measurements, wherein N is an integer greater than zero; and estimating, based on the N first BTTs, estimates of arterial deceleration times (ADTs) or venous acceleration times (VATs) in the target.

### CROSS-REFERENCE TO RELATED APPLICATION

This application claims priority to U.S. Provisional Application No. 63/663,579, filed on Jun. 24, 2024, the disclosure of which is hereby incorporated by reference herein in its entirety.

### TECHNICAL FIELD

This patent document relates to systems, devices, and processes for magnetic resonance imaging (MRI).

### BACKGROUND

Imaging through magnetic resonance imaging techniques has been widely applied in imaging applications in medical, biological and other fields. Current MRI methods that are capable of measuring vascular compliance are based on measuring the deformation (size change) of vessels at different cardiac phase or measuring blood velocity patterns using phase-contrast MRI. Both methods rely on resolving the vessels on MR images. Due to the limited spatial resolution of MRI within a reasonable imaging time, the two methods are limited to observation in relatively large vessels that are far from the brain tissue potentially affected by these vessels.

### SUMMARY

Techniques, systems and apparatus are described for a magnetic resonance imaging (MRI) system that allows measurement of arterial deceleration or venous acceleration time.

In one example aspect, a disclosed method includes operating the MRI system to make N first measurements of N first blood transit times (BTTs) in a target, wherein the N first measurements use a first value of an imaging cutoff velocity and N second values as labeling cutoff velocities respectively for the N first measurements, wherein Nis an integer greater than zero, and estimating, based on the N first BTTs, estimates of arterial deceleration times (ADTs) or venous acceleration times (VATs) in the target.

In another example aspect, a method of generating diagnostic data of a target object is disclosed. The method includes obtaining multiple MRI measurements of the target object; determining an ADT or a VAT of the target object based on the MRI measurements; and performing a clinical status determination of the target object based on the ADT and/or the VAT.

In another example aspect, an MRI system includes: a magnet; one or more radio frequency (RF) coils; one or more processors, wherein the one or more processors are configured to operate the MRI system to perform and above-described method.

In yet another aspect, a computer readable medium having processor-executable code is disclosed. The code, upon execution by one or more processors, causes the one or more processors to implement, by an MRI system, an above-described method.

In another aspect, a computer-readable storage medium having code stored thereupon, the code, upon execution by one or more processors, causing the one or more processors to implement a method recited in any of above method claims is disclosed.

These and other aspects and associated implementations and benefits of the disclosed technology are described in greater detail in the drawings, the description and the claims.

### BRIEF DESCRIPTION OF THE DRAWINGS

FIG. 1 is a flowchart of an example method of operating an MRI system.

FIG. 2 is a flowchart of an example method of operating an MRI system.

FIG. 3 is a block diagram of an example MRI system.

FIG. 4A is a diagram of a typical velocity-selective arterial spin labeling (VSASL) measurement.

FIG. 4B is a diagram showing an example technique used in performing an arterial deceleration time (ADT) measurement.

FIGS. 5-8 show example results obtained using single-module velocity-selective inversion (sm-VSI) in an experiment.

FIGS. 9-12 show example results obtained using dual-module velocity-selective inversion (dm-VSI) in an experiment.

FIGS. 13-16 show example results obtained using dual-module saturation pulses (dm-VSS) in an experiment.

### DETAILED DESCRIPTION

The present document discloses a new diagnostic technique that may be used by magnetic resonance imaging systems.

### Introduction

Being able to evaluate vascular health at the arteriole/capillary level is very challenging and may potentially provide valuable clinical information. Current MRI methods that are capable of measuring vascular compliance are based on measuring the deformation (size change) of vessels at different cardiac phase or measuring blood velocity patterns using phase-contrast MRI. Both methods rely on resolving the vessels on MR images. Due to the limited spatial resolution of MRI within a reasonable imaging time, the two methods are limited to observation in relatively large vessels that are far from the brain tissue potentially affected by these vessels.

In arterial spin labeling (ASL), arterial transit time (ATT) is the time needed for the labeled blood to travel from the labeled site to the destination tissue where the blook die perfusing to. In spatially selective methods, ATT (typically around 1 second) is mostly weighted by the portion the arterial blood travels in vessels in relatively large sizes. Various neurovascular diseases are directly affected by the health and function of the vessels at such microvascular level, for example, in Alzheimer's, vascular dementia, stroke and white matter diseases. Being able to evaluate vascular health at the arteriole/capillary level is very challenging but would provide valuable clinical information. In general, arterial blood decelerates as it enters smaller vessels, especially capillaries, but how fast it decelerates in this process, is largely unknown and very challenging to measure using MRI due to the small size. In recent years, more and more evidence supports the hypothesis that vascular compliance is a useful parameter to assess the health of the vascular health, e.g. more compliant vessels (softer) protect the brain tissue by dampening the pulsatile impact from the blood, and that stiffer vessels have indications of current vascular lesions or downstream events.

Some methods that are capable of measuring such flow dynamics include Doppler ultrasound and optical imaging. However, both modalities have significantly limited penetration and coverage in human applications, for example, they are not suitable for measuring the flow dynamics in the human brain which is well-protected by the skull.

As a result, currently there is no existing MRI method capable of providing the above-discussed diagnostic insights. The present document discloses methods that allow technicians to directly measure the deceleration or acceleration at the microvascular level, e.g., at arterioles, capillaries and venules, using MRI. Such blood flow dynamic parameters may provide valuable information on the health of the vessels at the microvascular level, which is close to and have direct impact on the health and the function of the brain tissue.

### Discussion

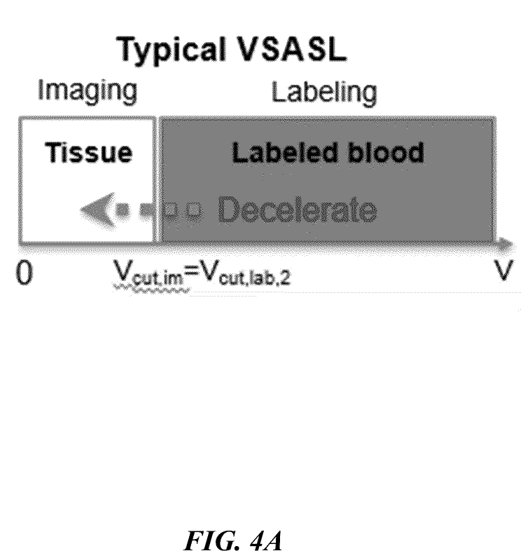

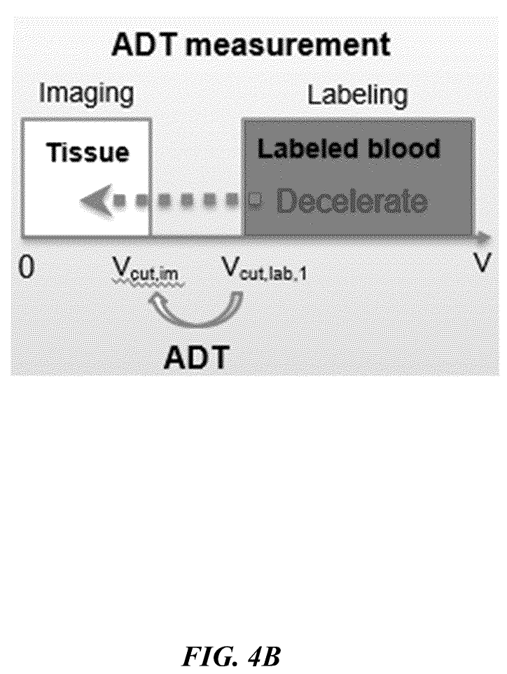

FIG. 4A shows an example of a typical velocity-selective arterial spin labeling measurement. In typical VSASL, the cutoff velocities (V_{cut}) in ASL signal preparation and image acquisition (V_{cut,im}) are matched, so that the labeled blood experiences effectively zero delay when it crosses this velocity boundary, i.e., decelerates on the way to tissue voxels, and the delivered arterial blood can be quantified. In contrast, the presently disclosed method uses two different V_{cut} at preparing and imaging the labeled blood.

FIG. 4B discloses an example technique of using two different V_{cut} in preparing and imaging the labeled blood. Taking the example of arterial deceleration time measurement, a V_{cut} is used in preparing (labeling, V_{cut,lab}) arterial blood such that the blood moving at higher velocities (V>V_{cut,lab}) is labeled, and a lower V_{cut} is used at image acquisition (V_{cut,im}), where (V_{cut,im}<V_{cut,lab}), now that the labeled arterial blood has to decelerate from V_{cut,lab} to V_{cut,im} to be measured, and the time it takes is the ADT. As can be seen in FIG. 4A, there is no separation between the velocity of labeling and imaging. However, FIG. 4B shows that the labeling velocity is above the imaging cutoff velocity. The difference between the two cutoff velocities is related to ADT. To measure the ADT, a multi-delay experiment can be performed with different delays after the labeling pulse to capture the inflow dynamics of the ASL signal, and the multi-delay VSASL signal can be fitted to a kinetic ASL signal model to estimate the delay time.

In principle, since the delay is effectively zero when V_{cut,lab}=V_{cut,im}, a direct measurement of arterial transit time (ATT) using V_{cut,lab}>V_{cut,im} is in fact ADT (decelerated from V_{cut,lab} to V_{cut,im}). However, in practice, due to the noise and potential artifacts in the measurement, more accurate results can be obtained by performing two sets of experiments: 1) ATT₁ measured with V_{cut,lab}=V_{cut,im}, and 2) ATT₂ measured with V′_{cut,lab}>V_{cut,lab}=V_{cut,im}; and the ADT can be estimated with ATT₂−ATT₁, so that the estimate bias caused by noise/artifact in ATT measurement can be subtracted out, leading to more accurate ADT estimates. In an example measurement strategy, two multi-delay VSASL ATT experiments can be performed as follows, with reference to FIGS. 4A-4B: 1) ATTᵥ₁ measured with V_{cut,lab,1}>V_{cut,im}, and 2) ATTᵥ₂ with V_{cut,lab,2}=V_{cut,im}. Arterial deceleration time can be measured as ADT=ATTᵥ₁−ATTᵥ₂, where V_{cut,lab,1}=V₁>V₂=V_{cut,lab,2}. The disclosed measurement strategy can lead to reduced error. Additionally, V₁ and V₂ can be selected to be sensitive to features such as arterioles/capillaries.

The VS labeling in the ADT mapping can be carried out by either VS saturation (VSS) or inversion (VSI) pulses. One important consideration is to reduce artifacts or errors at short delay times, where the artifacts tend to be strong. One option is to use VSS pulses that are robust against field inhomogeneities, such as symmetric BIR8 VSS pulse. Though VSI pulses are less robust against field inhomogeneities, they are advantageous for their higher SNR. Both VSS and VSI pulses can be used with the dual-module implementation to further reduce artifacts and errors, such as demonstrated in the in vivo examples disclosed herein.

Similar principles can be applied to measure venous acceleration time (VAT), where V_{cut,lab}<V_{cut,im} and an image acquisition to capture accelerated blood signal, such as velocity-selective excitation imaging method, can be used. The VAT may provide valuable information on the venous side of the microvasculature.

### One Example Implementation

In an example experiment, a healthy subject was studied using a 3T scanner (Siemens Prisma. The following parameters were implemented in the experimental setup: 3D GRASE EPI with field of view FOV=220×220 mm (64×64), 24 slices and 4 mm thickness; V_{cut,lab,1}=4 cm/s and V_{cut,lab,2}=2 cm/s, V_{cut,im}=2 cm/s in both; 16 pairs of label/control at each delay; variable TR with a fixed delay of 2 s before VSASL pulse. A two-step measurement strategy was employed: 1) single-module velocity-selective inversion with sinc modulation was used with TI=0.5 s, 1.0 s, 1.6 s, and 2.2 s, and a post-label delay (PLD) of 25 ms and 2) dual-module VSI with TI₁=0.2 s, 0.4 s, 0.8 s, and 1.4 s, and TI₂=50 ms. The same subject was studied in a third experiment performed at a later time using dual-module sym-BIR8 VSS with TI₁=0.5 s, 0.8 s, and 1.4 s, and TI₂=0 ms. Data processing was performed using pair-wise subtraction and averaging, no diffusion attenuation correction, fitting to the kinetic ASL signal model with the ATT allowed to be negative, and fitting performed voxel-wise and in gray matter (GM) and white matter (WM) region of interest (ROI).

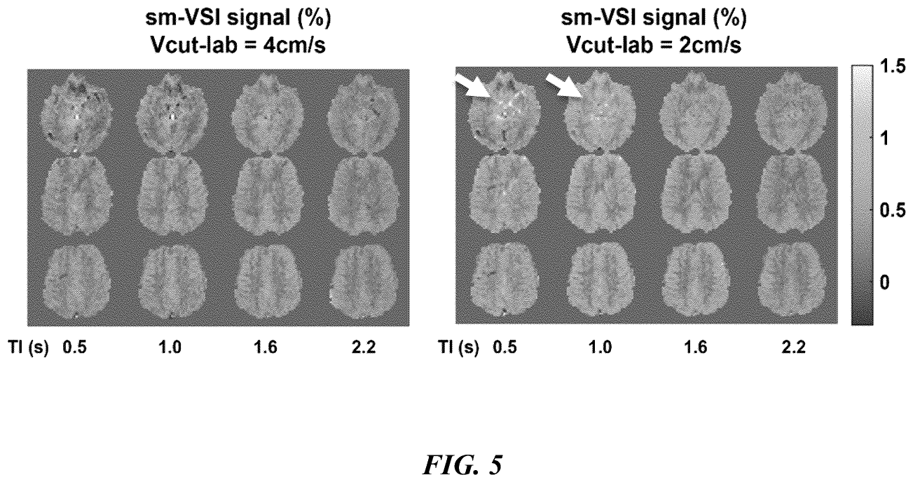

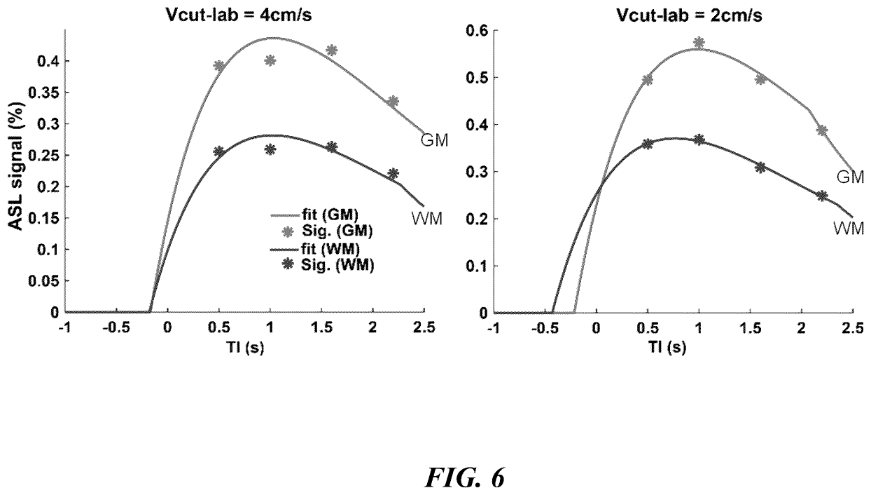

The results using sm-VSI are shown in FIGS. 5-8. FIG. 5 shows the sm-VSI signal at different TIs with V_{cut,lab}=4 cm/s (left) and 2 cm/s (right) in labeling. FIG. 6 shows example ROI fitting results for V_{cut,lab}=4 cm/s (left) and 2 cm/s (right). For V_{cut,lab}=4 cm/s (left in FIG. 6), ATT values were −0.17 s for GM and −0.18 s for WM. For V_{cut,lab}=2 cm/s (right in FIG. 6), ATT values were −0.22 s for GM and −0.43 s for WM. ADT was 0.05 s for GM and 0.25 s for WM. The ASL signals with sm-VSI labeling were contaminated by artifacts at short delays (e.g., white arrows in FIG. 5), resulting in negative and less reliable ATT at both V_{cut,lab}. The median per-voxel ATT in GM and WM with sm-VSI was −0.18 s and −0.14 s at V_{cut,lab,1}=4 cm/s, and −0.07 s and −0.33 s at V_{cut,lab,1}=2 cm/s, corresponding to ADT of 0.11 s and 0.19 s in GM and WM, respectively.

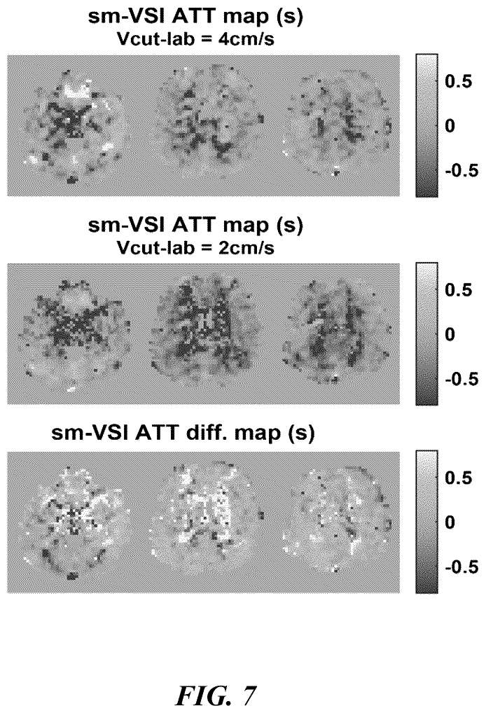

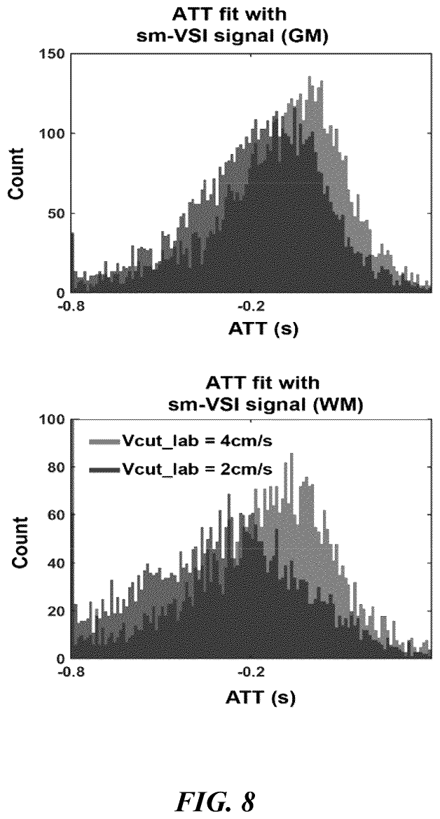

FIG. 7 shows examples of fitted ATT maps using sm-VSI signals for V_{cut,lab}=4 cm/s (top in FIG. 7) and V_{cut,lab}=2 cm/s (middle in FIG. 7), and the sm-VSI ATT difference (ADT) map (bottom in FIG. 7). FIG. 8 shows histograms of the fitted ATT in GM (top) and WM (bottom) voxels. Artifacts at early delays resulted in voxel-wise negative ATT values. ADT (ATT difference) can still be observed, but is likely biased. Table I shows example ATT and ADT values, for GM and WM, which were obtained from the experiment.

TABLE I
Example ATT and ADT values obtained using sm-VSI

| | ATT (s) | ATT (s) | ADT (s) |
|---|---|---|---|
| Median | V_{cut, lab} = 4 cm/s | V_{cut, lab} = 2 cm/s | 4 −> 2 cm/s |
| GM | −0.07 | −0.18 | 0.11 |
| WM | −0.14 | −0.33 | 0.19 |

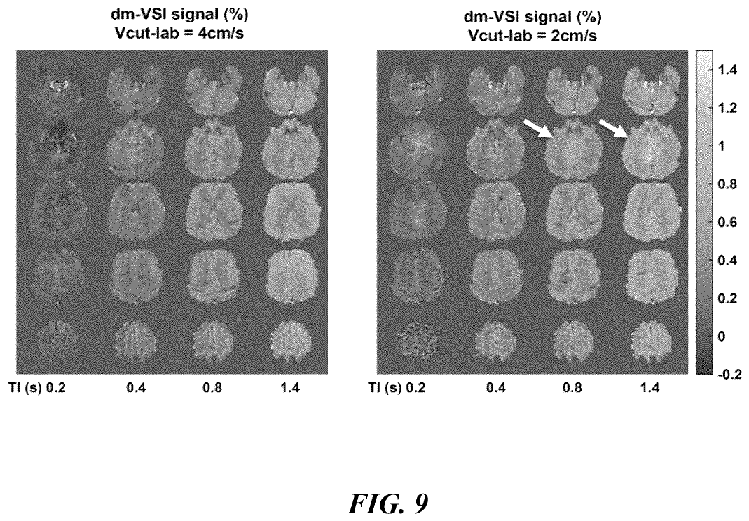

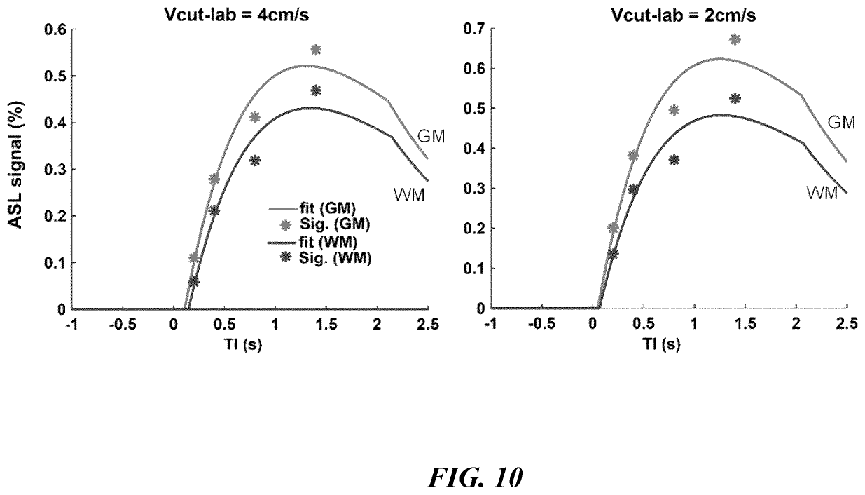

In contrast, much cleaner VSASL signals were observed at shorter delays with dm-VSI, indicating reduced artifacts and potentially more accurate ATT. The results using dm-VSI are shown in FIGS. 9-12. FIG. 9 shows the dm-VSI signal at different TIs with V_{cut,lab}=4 cm/s (left) and 2 cm/s (right) in labeling. FIG. 10 shows example dm-VSI ROI fitting results for V_{cut,lab}=4 cm/s (left) and 2 cm/s (right). For V_{cut,lab}=4 cm/s (left in FIG. 10), ATT values were 0.11 s for GM and 0.15 s for WM. For V_{cut,lab}=2 cm/s (right in FIG. 10), ATT values were 0.05 s for GM and 0.07 s for WM. ADT was 0.06 s for GM and 0.08 s for WM. FIG. 9 demonstrates the significant reduction of artifacts at early delays with dm-VSI labeling. Some artifacts are still present, e.g., at later delays as indicated with white arrows in FIG. 9. In FIG. 10, expected inflow dynamics can be observed and reasonable ATT values are achieved in ROI fitting. It can also be seen from FIG. 10 that ATT is close to zero when V_{cut,im}=V_{cut,lab}.

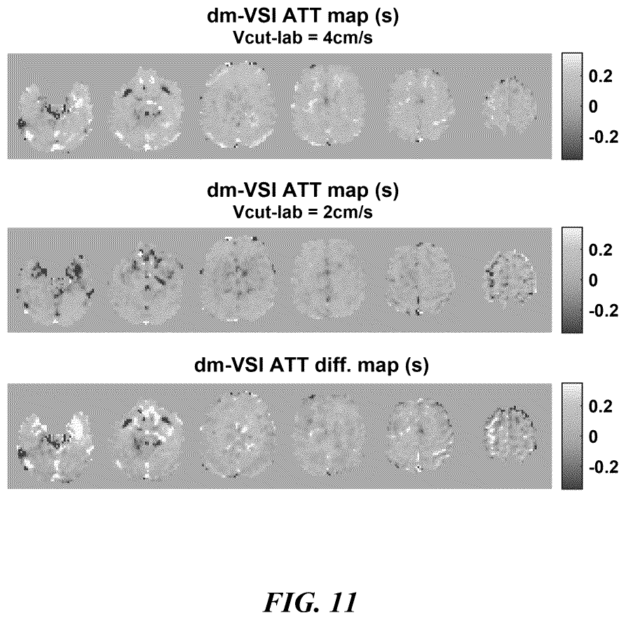

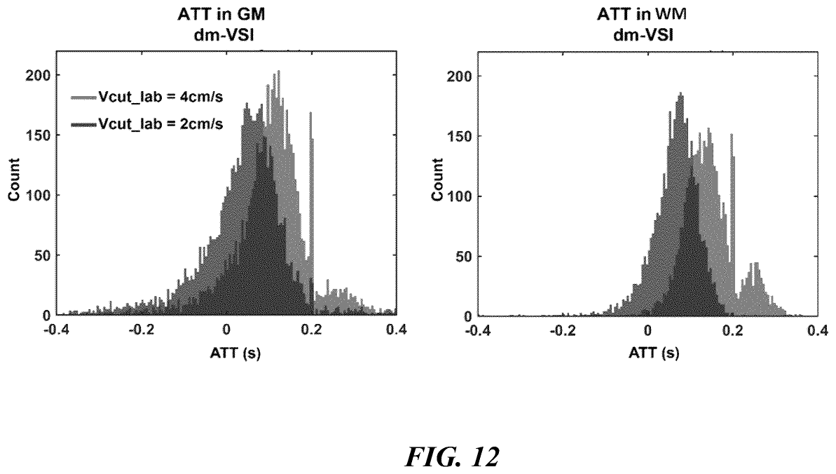

FIG. 11 shows examples of ATT and ADT maps using dm-VSI. FIG. 11 (top) shows ATT maps using dm-VSI signals for V_{cut,lab}=4 cm/s and V_{cut,lab}=2 cm/s (middle in FIG. 11), and the dm-VSI ATT difference (ADT) map (bottom in FIG. 11). FIG. 12 shows histograms of the fitted ATT in GM (left) and WM (right) for dm-VSI. Table II shows example ATT and ADT values, for GM and WM, which were obtained from the experiment using dm-VSI.

TABLE II
Example ATT and ADT values obtained using dm-VSI

| | ATT (s) | ATT (s) | ADT (s) |
|---|---|---|---|
| Median | V_{cut, lab} = 4 cm/s | V_{cut, lab} = 2 cm/s | 4 −> 2 cm/s |
| GM | 0.11 | 0.05 | 0.06 |
| WM | 0.14 | 0.07 | 0.07 |

As shown in Table II, the median ATT in GM and WM with dm-VSI was 0.11 s and 0.14 s at V_{cut,lab,1}=4 cm/s, and 0.05 s and 0.07 s at V_{cut,lab,2}=2 cm/s, corresponding to similar ADT of 0.06 s and 0.07 s in GM and WM, respectively.

It may be seen that, using the single-module method has early rising time, which is an artifact. However, the dual-module method provides a better control on artifacts. As can be seen in FIG. 9, left graph, even at 0.2 second, a difference can be seen between two curves. Dual module pulses therefore enable calculations of deceleration time in a finer way, e.g., for blood that reaches grey matter and white matter (e.g., less than 100 msec difference). FIG. 12 shows a histography with dm-VSI and the difference between two cutoff velocities. The data in FIG. 12 demonstrates that the blood at higher cutoff takes time to decelerate (curve shifted to right).

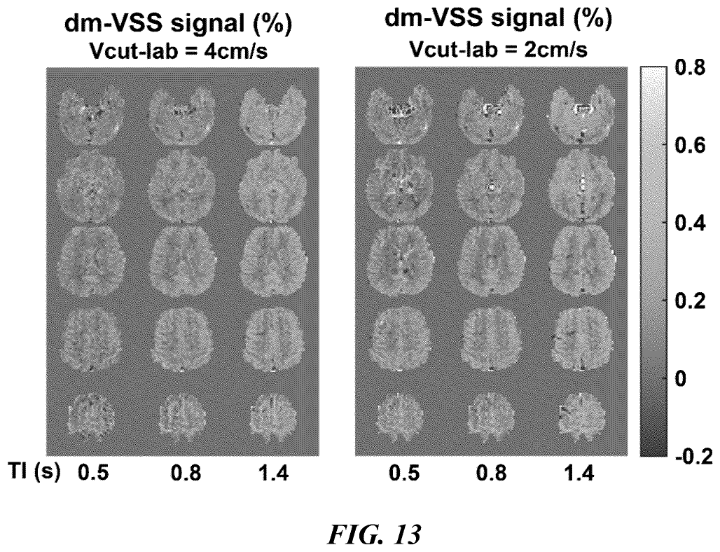

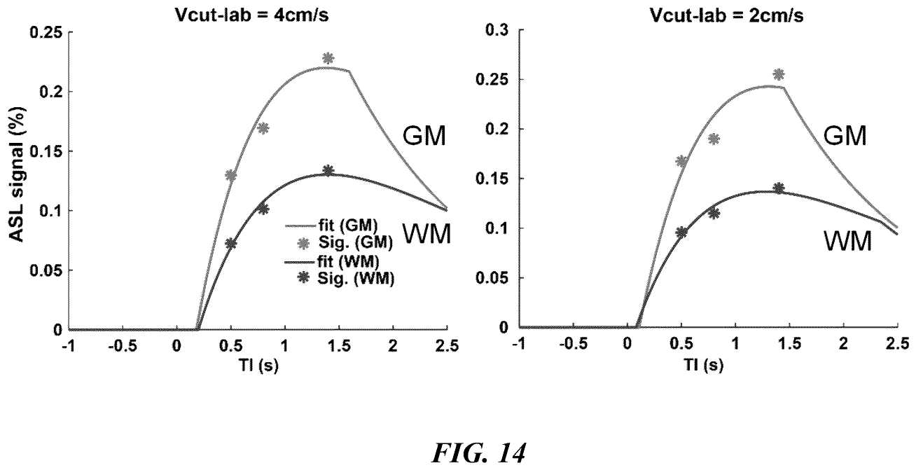

FIGS. 13-16 show example results when dual-module saturation pulses (dm-VSS) are used. Looking at the early inflow time, similar results can be seen at low time values (below 500 msec). FIG. 13 shows lower ASL signal compared to dm-VSI labeling and less artifacts at later delays than dm-VSI labeling. FIG. 14 shows example dm-VSS ROI fitting results for V_{cut,lab}=4 cm/s (left) and 2 cm/s (right). For V_{cut,lab}=4 cm/s (left in FIG. 14), ATT values were 0.18 s for GM and 0.20 s for WM. For V_{cut,lab}=2 cm/s (right in FIG. 14), ATT values were 0.11 s for GM and 0.08 s for GM. ADT was 0.07 s for GM and 0.12 s for WM. The results of FIG. 14 are similar to those using dm-VSI. It is observed that ATT is close to zero when V_{cut,im}=V_{cut,lab}, slightly longer than dm-VSI, and consistent with modeling prediction.

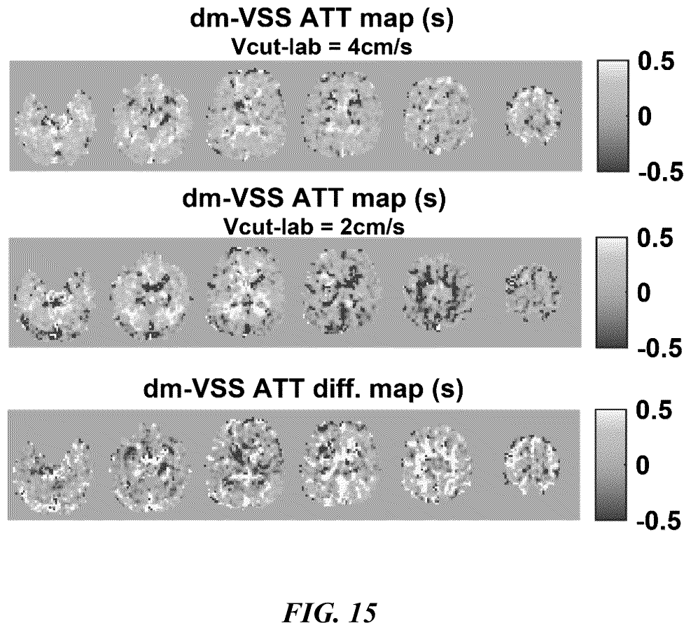

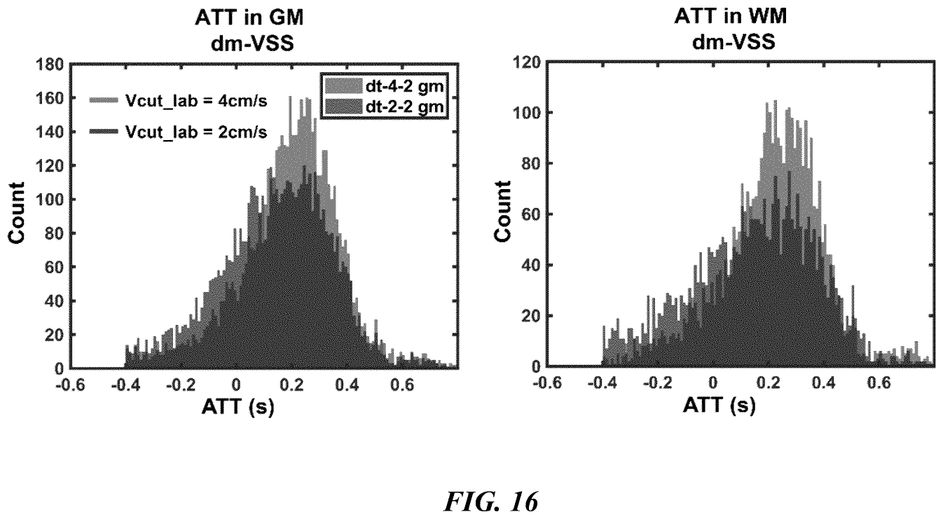

FIG. 15 shows examples of ATT and ADT maps using dm-VSS. FIG. 15 (top) shows ATT maps using dm-VSS signals for V_{cut,lab}=4 cm/s and V_{cut,lab}=2 cm/s (middle in FIG. 15), and the dm-VSS ATT difference (ADT) map (bottom in FIG. 15). FIG. 16 shows histograms of the fitted ATT in GM and WM for dm-VSS. Table III shows example ATT and ADT values, for GM (left) and WM (right), which were obtained from the experiment using dm-VSS. The results measured using dm-VSS are consistent with those measured using dm-VSI.

TABLE III
Example ATT and ADT values obtained using dm-VSS

| | ATT (s) | ATT (s) | ADT (s) |
|---|---|---|---|
| Median | V_{cut, lab} = 4 cm/s | V_{cut, lab} = 2 cm/s | 4 −> 2 cm/s |
| GM | 0.20 | 0.14 | 0.06 |
| WM | 0.22 | 0.13 | 0.09 |

Using techniques disclosed above, a direct measurement of ADT using MRI can be achieved. In experiments, it was demonstrated that the dm-VSASL helped reduce artifacts. Consistent results were obtained using dm-VSI and dm-VSS, and ATT insensitivity of VSASL with matched V_{cut,lab} and V_{cut,im} was confirmed.

Techniques disclosed above can also be implemented to measure venous acceleration time and offer potential clinical value in evaluating microvascular health.

### Clinical Use Examples

Using the above disclosed techniques, an MRI system may be able to acquire images and perform statistical analysis. The MRI system may perform the statistical analysis of the ADT values and provide the results to a clinical technician. The values being generated by ADT may be compared with a normalized set of values to get a better clinical understanding of the patient's situation. For example, the results obtained using the above techniques may be used against a know healthy range that is expected within the subject.

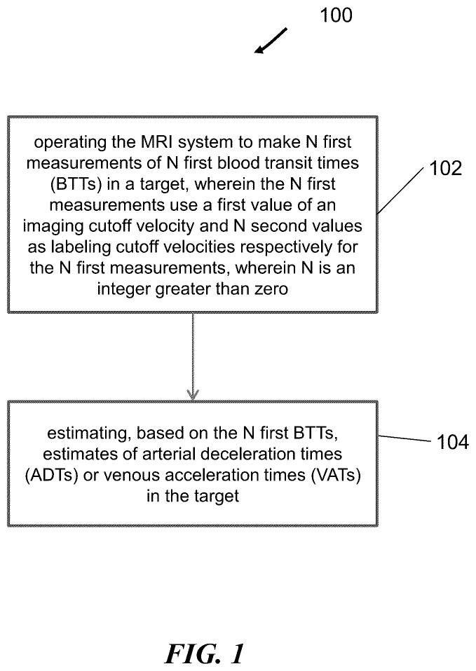

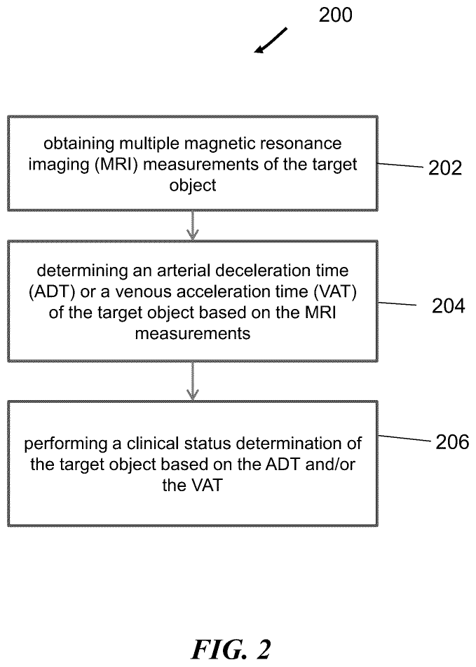

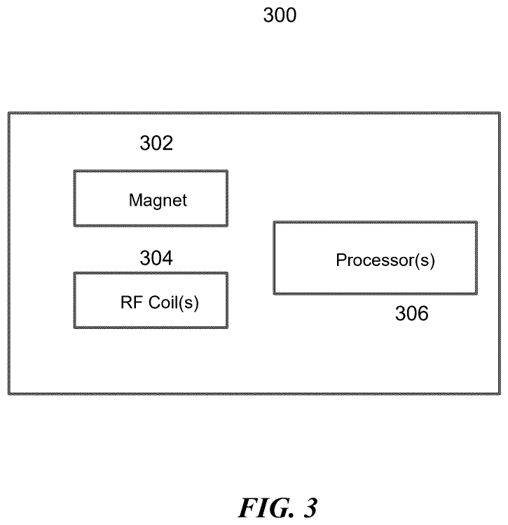

Some preferred embodiments may adopt the following technical solutions.
1. A method of operating a magnetic resonance imaging (MRI) system (e.g., method 100 depicted in FIG. 1), comprising: operating (102) the MRI system to make N first measurements of N first blood transit times (BTTs) in a target, wherein the N first measurements use a first value of an imaging cutoff velocity and N second values as labeling cutoff velocities respectively for the N first measurements, wherein N is an integer greater than zero; and estimating (104), based on the N first BTTs, estimates of arterial deceleration times (ADTs) or venous acceleration times (VATs) in the target.
2. The method of solution 1, wherein the N BTTs comprise arterial transit times (ATT) and/or venous transit times (VTT).
3. The method of solution 2, wherein the ADTs or VATs are equal to the ATTs or VTTs measured in the target, respectively.
4. The method of any of solutions 1-2, including: operating the MRI system to make a second measurement of a second BTT in a target, wherein the second measurement use the first value of imaging cutoff velocity and a third value of as a labeling cutoff velocity for the first measurement; and estimating, based on the N first BTTs and the second BTT, estimates of ADTs or VATs in the target.
5. The method of solution 4, wherein the third value is equal to the first value.
6. The method of any of solutions 1-5, wherein the N second values are dependent on a size of blood vessels at the target being subject to the first N measurements or the second measurement.
7. The method of any of above solutions, wherein the first value is determined based on an expected size of blood vessels at the target that are being subject to the N first measurements and/or the second measurement.
8. The method of any of above solutions, wherein the first measurement and the N second measurements are performed using a single-module velocity-selective pulse.
9. The method of any of solutions 1-7, wherein the N first measurements and the second measurement are performed using a dual-module velocity-selective pulse.
10. The method of solutions 8 or 9, wherein the velocity-selective pulse(s) are based on velocity-selective saturation (VSS) or velocity-selective inversion (VSI).
11. The method of solution 10, wherein the VSS pulse comprises a B1 insensitive rotation (BIR) pulse.
12. The method of solution 10, wherein the VSI pulse comprises a Fourier-Transform VSI pulse with a sinc modulation.
13. A magnetic resonance imaging (MRI) system (e.g., system 300 depicted in FIG. 3), comprising: a magnet (302); one or more radio frequency (RF) coils (304); one or more processors (306); and wherein the one or more processors are configured to operate the MRI system to perform the method of any of solutions 1-12.
14. A method of generating diagnostic data of a target object (e.g., method 200 depicted in FIG. 2), comprising: obtaining (202) multiple magnetic resonance imaging (MRI) measurements of the target object; determining (204) an arterial deceleration time (ADT) or a venous acceleration time (VAT) of the target object based on the MRI measurements; and performing (206) a clinical status determination of the target object based on the ADT and/or the VAT.
15. The method of solution 14, wherein the clinical status determination is performed by comparing the ADT or the VAT with an expected normal range for the target object.
16. A computing system for generating diagnostic data, comprising one or more processors configured to operate the computing system to perform a method of any of solutions 14-15.
17. A computer-readable storage medium having code stored thereupon, the code, upon execution by one or more processors, causing the one or more processors to implement a method recited in any of above method solutions.

### Conclusion

The embodiments disclosed herein include a new method that is the first to directly measure the acceleration and deceleration time characteristics of blood using MRI. In the context of measuring vessel properties such as vascular compliance, it has the following advantages in comparison to existing methods, for example, spatially-selective ASL based methods:
1) The disclosed method does not rely on resolving individual vessels, so it can provide better resolution and closer to capillaries/tissue, where existing methods can only be applied on large vessels and may not provide clinically relevant information at the capillary/tissue level;
2) The disclosed method makes a greater coverage available since it does not rely on measurement in the limited regions supplied by resolvable vessels;
3) The disclosed method is applicable on clinical MRI scanners of magnetic strengths, such as 1.5 or 3 Tesla, whereas ultra-high fields (e.g., 7 Tesla) are required for existing methods in order to resolve small sized vessels for regional measurement.

In comparison to ultrasound-based or optical methods, the disclosed method has higher spatial resolution, much deeper penetration (e.g., not obstructed by skull) and significantly greater coverage. This is the first measurement of ADT using MRI. The plausible preliminary results, especially that using dm-VSI, showing similar ADT in GM and WM as expected. The ATT measured with matched V_{cut,lab} and V_{cut,im} also confirmed the ATT-insensitivity of VSASL.

Correction for diffusion attenuation effects can be used to reduce the artifactual signals at short delays, which was not applied here. The dm-VSASL approach helped reduce artifacts without such correction, which is advantageous in mapping ATT and ADT in VSASL. However, there were artifacts at longer delays, which requires further investigation.

Similar experiments can be designed to measure the acceleration times on the venous side, i.e., venous acceleration time, using the principle of velocity-selective excitation to isolate the venous signals.

ADT may have values in evaluating microvascular health as a potential biomarker.

While this specification contains many specifics, these should not be construed as limitations on the scope of an invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or a variation of a subcombination.

The described systems, apparatus and techniques can be implemented in electronic circuitry, computer hardware, firmware, software, or in combinations of them, such as the structural means disclosed in this specification and structural equivalents thereof. This can include at least one computer-readable storage medium embodying a program operable to cause one or more data processing apparatus (e.g., a signal processing device including a programmable processor) to perform operations described. Thus, program implementations can be realized from a disclosed method, system, or apparatus, and apparatus implementations can be realized from a disclosed system, computer-readable medium, or method. Similarly, method implementations can be realized from a disclosed system, computer-readable medium, or apparatus, and system implementations can be realized from a disclosed method, computer-readable medium, or apparatus.

Only a few implementations are disclosed. However, variations and enhancements of the disclosed implementations and other implementations can be made based on what is described and illustrated in this specification.

1. A method of operating a magnetic resonance imaging (MRI) system, comprising:
operating the MRI system to make N first measurements of N first blood transit times (BTTs) in a target, wherein the N first measurements use a first value of an imaging cutoff velocity and N second values as labeling cutoff velocities respectively for the N first measurements, wherein N is an integer greater than zero; and
estimating, based on the N first BTTs, estimates of arterial deceleration times (ADTs) or venous acceleration times (VATs) in the target,
wherein the N BTTs comprise arterial transit times (ATT) or venous transit times (VTT).

2. The method of claim 1, wherein the ADTs or VATs are equal to the ATTs or VTTs measured in the target, respectively.

3. The method of claim 1, including:
operating the MRI system to make a second measurement of a second BTT in a target, wherein the second measurement use the first value of imaging cutoff velocity and a third value of as a labeling cutoff velocity for the first measurement; and
estimating, based on the N first BTTs and the second BTT, estimates of ADTs or VATs in the target.

4. The method of claim 3, wherein the third value is equal to the first value.

5. The method of any claim 1, wherein the N second values are dependent on a size of blood vessels at the target being subject to the first N measurements or the second measurement.

6. The method of claim 1, wherein the first value is determined based on an expected size of blood vessels at the target that are being subject to the N first measurements or the second measurement.

7. The method of any of claim 1, wherein the first measurement and the N second measurements are performed using a single-module velocity-selective pulse, wherein the N first measurements and the second measurement are performed using a dual-module velocity-selective pulse.

8. The method of claim 7, wherein the velocity-selective pulse(s) are based on velocity-selective saturation (VSS) or velocity-selective inversion (VSI).

9. The method of claim 8, wherein the VSS pulse comprises a B1 insensitive rotation (BIR) pulse, wherein the VSI pulse comprises a Fourier-Transform VSI pulse with a sinc modulation.

10. A magnetic resonance imaging (MRI) system, comprising:
a magnet;
one or more radio frequency (RF) coils;
one or more processors; and
wherein the one or more processors are configured to operate the MRI system to perform a method of operating a magnetic resonance imaging (MRI) system, comprising:
operating the MRI system to make N first measurements of N first blood transit times (BTTs) in a target, wherein the N first measurements use a first value of an imaging cutoff velocity and N second values as labeling cutoff velocities respectively for the N first measurements, wherein N is an integer greater than zero; and
estimating, based on the N first BTTs, estimates of arterial deceleration times (ADTs) or venous acceleration times (VATs) in the target,
wherein the N BTTs comprise arterial transit times (ATT) or venous transit times (VTT).

11. A method of generating diagnostic data of a target object, comprising:
obtaining multiple magnetic resonance imaging (MRI) measurements of the target object;
determining an arterial deceleration time (ADT) or a venous acceleration time (VAT) of the target object based on the MRI measurements; and
performing a clinical status determination of the target object based on the ADT and/or the VAT.

12. The method of claim 11, wherein the clinical status determination is performed by comparing the ADT or the VAT with an expected normal range for the target object.

13. A computing system for generating diagnostic data, comprising one or more processors configured to operate the computing system to perform a method of generating diagnostic data of a target object, comprising:
obtaining multiple magnetic resonance imaging (MRI) measurements of the target object;
determining an arterial deceleration time (ADT) or a venous acceleration time (VAT) of the target object based on the MRI measurements; and
performing a clinical status determination of the target object based on the ADT and/or the VAT.

14. The computing system of claim 13, wherein the clinical status determination is performed by comparing the ADT or the VAT with an expected normal range for the target object.

15. A computer-readable storage medium having code stored thereupon, the code, upon execution by one or more processors, causing the one or more processors to implement a method of operating a magnetic resonance imaging (MRI) system, comprising:
operating the MRI system to make N first measurements of N first blood transit times (BTTs) in a target, wherein the N first measurements use a first value of an imaging cutoff velocity and N second values as labeling cutoff velocities respectively for the N first measurements, wherein N is an integer greater than zero; and
estimating, based on the N first BTTs, estimates of arterial deceleration times (ADTs) or venous acceleration times (VATs) in the target,
wherein the N BTTs comprise arterial transit times (ATT) or venous transit times (VTT).

16. The computer-readable storage medium of claim 15, wherein the ADTs or VATs are equal to the ATTs or VTTs measured in the target, respectively.

17. The computer-readable storage medium of claim 15, wherein the method further comprises:
operating the MRI system to make a second measurement of a second BTT in a target, wherein the second measurement use the first value of imaging cutoff velocity and a third value of as a labeling cutoff velocity for the first measurement; and
estimating, based on the N first BTTs and the second BTT, estimates of ADTs or VATs in the target.

18. The computer-readable storage medium of claim 15, wherein the N second values are dependent on a size of blood vessels at the target being subject to the first N measurements or the second measurement, wherein the first value is determined based on an expected size of blood vessels at the target that are being subject to the N first measurements or the second measurement.

19. A computer-readable storage medium having code stored thereupon, the code, upon execution by one or more processors, causing the one or more processors to implement a method of generating diagnostic data of a target object, comprising:
obtaining multiple magnetic resonance imaging (MRI) measurements of the target object;
determining an arterial deceleration time (ADT) or a venous acceleration time (VAT) of the target object based on the MRI measurements; and
performing a clinical status determination of the target object based on the ADT and/or the VAT.

20. The computer-readable storage medium of claim 19, wherein the clinical status determination is performed by comparing the ADT or the VAT with an expected normal range for the target object.
